(19) United States
(12) Patent Application Publication
(10) Pub. No.: US 2017/0273881 A1
(43) Pub. Date: Sep. 28, 2017

(54) COSMETIC COMPOSITION FOR KERATIN FIBRES

(71) Applicant: Alfa Parf Group S.P.A., Bologna (IT)

(72) Inventors: Francesco Facheris, Dalmine (bg) (IT); Gianluca Borgnini, Carvico (bg) (IT); Antonio Consoli, Urgnano (bg) (IT); Katiuscia Grevalcuore, Bergamo (IT)

(21) Appl. No.: 15/504,052

(22) PCT Filed: Aug. 6, 2015
(86) PCT No.: PCT/EP2015/068181

§ 371 Date: Feb. 15, 2017

(30) Foreign Application Priority Data
Aug. 21, 2014 (IT) MI2014A001508

(51) Int. Cl. A61K 8/36; A61K 8/04; A61Q 5/04; A61Q 5/06; A61K 8/44

(52) U.S. Cl. CPC A61K 8/36; A61Q 5/06; A61K 8/44; A61Q 5/04; A61K 8/046

(57) ABSTRACT

The present invention relates to a cosmetic composition based on glyoxylic acid and/or derivatives in foam form able to reshape the form of keratin fibre, and a method of reshaping the form of keratin fibre with said composition.

### TECHNICAL FIELD OF INVENTION

The present invention relates to a cosmetic composition based on glyoxylic acid and/or derivatives thereof able to reshape the form of keratin fibre, in particular human keratin fibre such as hair. The present composition takes the form of a foam, obtained with a particular non-aerosol device. The invention also relates to a method of reshaping hair using the cosmetic composition.

### PRIOR ART

The cosmetic industry offers a large number products of different types for beautifying the hair. Said products are now commonly used in everyday life. In addition to common treatments, which include products like shampoo, styling products and dyes, there are also hair reshaping products which are becoming one of the most important treatments performed by hairdressers. The need arises from the fact that people wish to have straight or curly hair depending on the current fashion, and at the same time to facilitate everyday styling. For this purpose the market currently offers various products able to change the natural form of the hair, some of which are able to vary its form from straight to curly/wavy, and others vice versa. The latter are also known as smoothing treatments.

One of the major problems associated with the majority of compounds used to straighten wavy, curly, frizzy or kinky hair is that said compounds are highly aggressive towards the hair.

Three general hair reshaping methods have been described over the years. The first two involve significant cleavage of a considerable percentage of the disulphide bridges deriving from cystine residues. Opening of the disulphide bridges is generally associated with mechanical reshaping of the hair, such as that performed by a comb. To change the form of the hair it is necessary to change the relative positions of two adjacent polypeptide chains of the keratin fibre.

The third general method is represented by the introduction of a bridge bond with the aid of aldehydes.

Method 1: Use of Reducing Agents

This method involves a chemical reduction of the cystine structure into two cysteine units, followed by reconfiguration of the hair fibres and re-formation of the cystine structure by oxidation.

The —S—S— bonds of keratin fibre generally maintain the hair in its natural straight or curly configuration. In order to reshape the hair permanently into a different configuration, the disulphide bonds (—S—S—) must be cleaved in significant quantities to form two —SH groups. At this stage, the protein chains of the hair are detached from one another, and the hair may be reshaped with the use of suitable supports. Subsequently, by applying hydrogen peroxide, new disulphide bridges are formed. The formation of the new —S—S— bonds has a lasting effect on the shape of the hair in the new configuration. The main ingredient used in this first method of reducing the cystine bonds is ammonium thioglycolate.

Bisulphite and/or sulphite solutions were once, and sometimes still are, used to cleave the disulphide bridges into thiol groups and form a Bunte salt; this method was discovered by Clark and Speakmannel in 1932 (see W. Umbach: Kosmetik—Entwicklung, Herstellungund AnwendungkosmetischerMittel, 2nd edition, Georg ThiemeVerlag, Stuttgart, 1995). The reducing composition, which contains a large amount of bisulphite and/or sulphite, does not have an intrinsic odour like that of mercaptans.

Fixing is also supported with the aid of heat or oxidising agents, which lead to the formation of a new disulphide bridge. In any event, the degree of hair damage is high. For this reason, and also for safety reasons, this method is no longer often used.

One of the disadvantages of using reducing agents is that after their application, an oxidising agent such as hydrogen peroxide is used to reconnect the disulphide bridges and deactivate the reducing agent.

Reducing compounds are normally used in an alkaline environment; any excess hydrogen peroxide must therefore be thoroughly removed to prevent hair colour loss problems. Loss of colour may be observed if the hair has previously been artificially dyed with a composition containing ammonium thioglycolate.

Method 2: Use of Alkaline Agents

Metal hydroxides were used for many years as an efficient hair straightening method. Sodium hydroxide is commonly used as a chemical straightener, and gives a permanent effect over time. Alkalis (sodium hydroxide, potassium hydroxide and lithium hydroxide) are key ingredients of products called “lyes”.

In U.S. Pat. No. 4,304,244, guanidine hydroxide is mentioned as another option for chemical hair straightening; straighteners containing said compound are normally called “no-lye” straighteners.

In any event, both types of straightener have a pH ranging between 12 and 14.

Hair treatments containing strong bases like the above-mentioned hydroxides possess an action mechanism comprising two steps. The first step leads to cleavage of the (—S—S—) cystine bridges, with formation of cysteine on the one hand and dehydroalanine on the other.

According to C. Zviak, The Science of Hair Care, pp. 185-186 (1986), a sulphur atom is removed from the hair and converted to an HOS ion⁻. In this situation most of the polypeptide chains are not bonded together, and the hair may be reshaped at this stage. In the second step the cysteine groups react with the double bond of the dehydroalanine residue to form a lanthionine bridge (—CH2-S—CH2-), which reconnects the polypeptide chains.

Treatment with hydroxides, in particular alkaline earth hydroxides, leads to the formation of stable, irreversible bonds, with the result that it is impossible to perform a subsequent treatment to change the form of the hair.

Moreover, said chemical composition is very aggressive to the skin of the scalp and to the hair. Even if scalp protectors are used, said compositions can cause strong irritation. A reduction in the elasticity of the hair and, in some extreme cases, even hair loss, therefore results. Lengthy exposure to strong alkaline agents can also dissolve the hair. A pre-application diagnosis to establish the health of the hair, and continuous monitoring during the processing time, should therefore be considered.

Various attempts were therefore made to replace hydroxides with ingredients that are less aggressive to the hair. In any event, said compositions were unsatisfactory, in terms of both their straightening effect and their cosmetic effect.

Method 3: Use of Formaldehyde

U.S. Pat. No. 2,390,073 mentions a hair treatment based on the use of formaldehyde or a compound able to release it. According to said document, formaldehyde creates new bridges in the polypeptide structure, which increase the strength of the natural inter-chain bond between two cysteines and generate the straightening effect. The typical concentration used in these types of straightener ranges from 5 to 10% v/v. Subsequently, formaldehyde adducts were marketed in various straightening compositions until the use of formaldehyde was regulated and its maximum concentration was set at 0.2% v/v for safety reasons. A considerable amount of formaldehyde evaporates during treatment, because the process requires a hairdryer or hair iron which facilitates the reaction that forms the inter-chain bonds and gives the hair its new form. In view of the toxicological profile of formaldehyde and its adducts, it was concluded that said products do not possess the required safety levels.

US 2013/0118520A1 states that human hair can also be reshaped with products containing high concentrations of glyoxylic acid at high temperatures, using a hair iron. The pH range of said products is normally between 0.8 and 3.0. The drawback of using glyoxylic acid in combination with the high temperatures required by the treatment is an evident colour fade of the hair, especially if artificially coloured, which leads to customer dissatisfaction.

The cosmetic forms currently available on the market which use glyoxylic acid are solutions, fluid emulsions, creams and foams produced with a non-aerosol pump-driven foamer. Foams allow more even distribution of the product on the hair than other forms.

Pump-driven foamers contain internal metal parts which are incompatible with the acid operating environment of glyoxylic acid. Said devices must also contain a low-viscosity formulation to allow correct foam dispensing and formation without the addition of viscosity-controlling agents.

Said formulation involves problems of dripping during application to the hair.

Said pumped foams also present drawbacks when applied to the hair, because the pump must be continually activated whenever foam needs to be generated; the amount generated by each squeeze of the pump is limited, which considerably slows application to the hair; it is also necessary to put down the container whenever two hands are needed to distribute the product as evenly and quickly as possible on the hair.

The objective of the present invention is therefore to formulate a cosmetic composition that eliminates the drawbacks of the existing compositions, and is not only innocuous but also safe for use by professional users and for customers.

### SUMMARY OF THE INVENTION

The present invention relates to a cosmetic composition for semi-permanent hair straightening or semi-permanent hair reshaping comprising:
glyoxylic acid and/or at least one derivative of formula (I), (II) or (III) as defined below, ranging from 0.5% to 30% by weight of the total weight of the composition,
at least one foaming agent selected from anionic, cationic and non-ionic surfactants, and
at least one viscosity-controlling agent to obtain a viscosity ranging from 200 to 3000 cP,
   in the form of a foam.

The present invention relates to a cosmetic composition in the form of a foam obtainable after shaking a composition comprising glyoxylic acid and/or at least one derivative of formula (I), (II) or (III) as defined below; at least one foaming agent selected from anionic, cationic and non-ionic surfactants; and at least one viscosity-controlling agent, with a viscosity ranging between 200 and 3000 cP, in a device having a ratio between outlet hole height and diameter of less than 3:1.

The invention also relates to the use of the cosmetic composition to semi-permanently reshape or straight the hair.

The invention further relates to a method for semi-permanent hair straightening or semi-permanent hair reshaping with the cosmetic composition according to the invention.

### DETAILED DESCRIPTION OF THE INVENTION

It has surprisingly been found that a cosmetic composition in the form of a foam is obtained from a cosmetic composition containing glyoxylic acid and/or at least one derivative of formula (I), (II) or (III) as defined below, at least one foaming agent selected from anionic, cationic and non-ionic surfactants, and at least one viscosity-controlling agent, with a viscosity ranging between 200 and 3000 cP (measured with a Brookfield RVII with spindle 3 at the speed of 10 rpm at the temperature of 25° C.), after shaking in a device having a ratio between outlet hole height and diameter of less than 3:1. The use of said foam accelerates application to the hair, because it is always available throughout the processing time; it does not drip from the hand during application and processing; and its production does not require a sophisticated device with a specific foamer which may have internal metal parts liable to corrosion problems.

According to a preferred aspect of the invention, the at least one derivative has structural formula (I), (II) or (III):

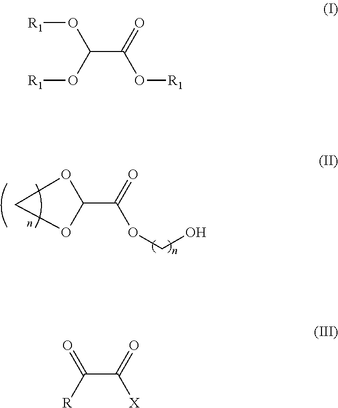

In structural formula (I), R1 can be selected from H, a C1-C8 alkyl group, a C1-C8 monohydroxyalkyl group, or a C3 or C4 dihydroxyalkyl group.

In structural formula (II), n is 2 or 3.

In structural formula (III), R is H or —CH₃ and X is —OH or —NHR₂, wherein R₂ represents an amino-acid radical and N is the amino group corresponding to the amino-acid group.

According to a further preferred aspect of the invention, in structural formula (III), R is H and X is —OH or —NHR₂ wherein R₂ is as defined above.

According to a further preferred aspect of the invention, in structural formula (III), R is —CH₃ and X is —OH.

Derivatives of glyoxylic acid with amino acids which have the carbonamide structure are generally called glyoxyloyl amino acids. Amino acids usable to form the derivatives of glyoxylic acid with amino acids having structural formula (IV) include glycine, aspartic acid, glutamic acid, cysteic acid, cystine and carbocysteine. Glyoxyloyl carbocysteine, indicated by the INCI name of Glyoxyloyl Carbocysteine (INCI—International Nomenclature of Cosmetic Ingredients: Decision of the European Community 96/335/EC as amended by 2006/257/EC), is the preferred derivative according to the present invention.

Glyoxylic acid or at least one derivative or mixtures thereof is present in the cosmetic composition in a concentration sufficient to perform the reshaping effect on the keratin fibre without damaging the fibre.

The concentration may range from 0.5% to 30% by weight of the total weight of the composition, preferably from 5% to 25% by weight.

The composition also includes a foaming agent selected from cationic surfactants, anionic surfactants, non-ionic surfactants, amphoteric surfactants and mixtures thereof. The use concentration of the single surfactant or combination thereof may range from 0.5% to 30% by weight of the total composition, preferably from 1% to 10% by weight.

The non-ionic surfactants usable may be surfactants containing a lipophilic chain, preferably a C8-22 linear alkyl or acyl chain and a hydrophilic group such as a glucoside or polyglucoside, a glycerol or polyglycerol, a sorbitan or polyglycol ether, or several of said groups. The non-ionic surfactants usable may also be all those produced by adding fatty alcohols, fatty acids, mono- or diglyceride fatty acids, alkanolamides of fatty acids, esters of fatty acids and sorbitan, esters of fatty acids and methyl glucoside, or alkyl glycosides, to ethylene oxide. Examples of usable non-ionic surfactants are ethoxylates of fatty alcohol such as PEG-40 castor oil (reported with the INCI name). The non-ionic surfactants usable (reported with the INCI name) are preferably Coco Glucoside, Decyl Glucoside and Lauryl Glucoside.

These surfactants may be present in the composition from 0.5% to 30% by weight of the total weight of the composition, preferably from about 1% to 10%.

The anionic surfactants usable may be surfactants preferably characterised by an aliphatic linear chain containing up to 5-6 carbon atoms comprising a sulphate, sulphonate, phosphate or carboxyl group. In particular, they may be selected from alkyl sulphates, alkylethersulphates, alkylarylsulsphonates, alphaolefinsulphonates, acylisethionates, acyltaurates, acylsarcosinates, sulphosuccinates, alkoylpolypeptides, acylglutamates, citric and tartaric acid derivatives, carboxylated alkylethers in their alkaline earth metal salt form, magnesium, ammonium or alkanolamines.

Anionic surfactants may also be included in the cosmetic composition in solid phase.

The anionic surfactants used are preferably selected from Sodium Lauryl Sulfate, Sodium Laureth Sulfate and Ammonium Laureth Sulfate (the INCI names is given).

These surfactants may be present in the composition from 0.5% to 30% by weight of the total weight of the composition, preferably from about 1% to 10%.

The amphoteric surfactants usable may be selected from alkyl betaine, alkylamidopropyl betaine, amphoacetates, amphodiacetates and propionates. The amphoteric surfactants usable are preferably cocoamidopropyl betaine, available under the trade name TegoBetaine CKD (N—N-dimethyl-N-lauroylamidopropyl)-ammoniumacetobetaine), sodium cocoamphoacetate (INCI name: Sodium Cocoamphoacetate) available under the trade name DEHYTON G, and disodium dicocoamphoacetate (INCI name Disodium Cocoamphodiacetate), available under the trade name DEHYTON DC.

These surfactants may be present in the composition from 0.5% to 30% by weight of the total weight of the composition, preferably from about 1% to 10%.

The cationic surfactants usable are, for example, quaternary ammonium salts such as the following (reported with their chemical name or INCI name): (C10-C24)-alkyldimethylammonium chloride or bromide, preferably di(C12-C18)-alkyldimethylammonium chloride or bromide; (C10-C24)-alkyldimethylethylammonium chloride or bromide; (C10-C24)-alkyltrimethylammonium chloride or bromide, preferably cetyltrimethylammonium chloride or bromide; (C20-C22)-alkyltrimethylammonium chloride or bromide; (C10-C24)-alkyldimethylbenzylammonium chloride or bromide, preferably (C12-C18)-alkyldimethylbenzylammonium chloride; N—(C10-C18)-alkylpyridinium chloride or bromide, preferably N—(C12-C16)-alkylpyridinium chloride or bromide; N—(C10-C18)-alkylisoquinoline or monoalkylsulphate chloride or bromide; N—(C12-C18)-alkylpolyoylaminoformylmethylpyridinium chloride; and the following (INCI names given): N—(C12-C18)-alkyl-N-methylmorpholiniumchloride, bromide or monoalkylsulfate; N—(C12-C18)-alkyl-N-ethylmorpholiniumchloride, bromide, or monoalkylsulfate; (C16-C18)-alkylpentaoxyethylammoniumchloride; diisobutylphenoxyethoxyethyldimethylbenzylammoniumchloride; N,N-diethylamino-ethylstearylamide and -oleylamide salts and hydrochloric acid, acetic acid, lactic acid, citric acid and phosphoric acid; N-acylaminoethyl-N,N-diethyl-N-methylammoniumchloride, bromide or monoalkylsulfate and N-acylaminoethyl-N,N-diethyl-N-benzylammoniumchloride, bromide or monoalkylsulfate, wherein the acyl group is preferably stearyl or oleyl. Cetyltrimethylammoniumchloride, cetylpyridiniumchloride, benzalkoniumchloride, benzethoniumchloride, dimethyl-dioctadecylammoniumchloride, dioctadecyldimethylammoniumbromide and distearoyl-ethyldimoniumchloride (DSEDC) are preferred.

These surfactants may be present in the composition from 0.5% to 20% by weight of the total weight of the composition, preferably from about 1% to 10%.

The viscosity-controlling agent may be selected from non-ionic, cationic, anionic or amphoteric viscosity-controlling agents or mixtures thereof.

Said viscosity-controlling agents may be present in the composition in quantities ranging from 0.1% to 10% by weight of the total weight of the composition, preferably from about 0.2% to 5%.

The anionic viscosity-controlling agents usable comprise acryloyldimethyltaurate/VP copolymer, ammonium acryloyldimethyltaurate/carboxyethylacrylate crosspolymer and ammonium acryloyldimethyltaurate/beheneth-25 methacrylate crosspolymer.

The anionic viscosity-controlling agents may also be alginates, polysaccharides, carboxymethylcellulose, alginic acid, sodium alginate, ammonium alginate, calcium alginate, gum arabic, guar gum or xanthan gum, used individually or in combination with other anionic viscosity-controlling agents. According to the invention, xanthan gum or cellulose derivatives are preferably used, and more preferably xanthan gum.

The cationic viscosity-controlling agents usable may be those derived from hydroxypropyl guar, commonly identifiable with the INCI name of Hydroxypropyltrimonium Chloride and available on the market under the trade names Catinal CG-100 and Catinal CG-200 made by the company Toho, CosmediaGuar C-261N, CosmediaGuar C-261N and CosmediaGuar C-261N available from the company Cognis, DiaGum P 5070 from the company Freedom Chemical Diamalt, N-HanceCationicGuar available from the company Hercules/Aqualon, Hi-Care 1000, Jaguar C-17, Jaguar C-2000, Jaguar C-13S, Jaguar C-14S and Jaguar Excel available from the company Rhodia, Kiprogum CW and Kiprogum NGK available from Nippon Starch. This class also includes the hydroxypropyl derivatives of hydroxypropyltrimonium guar hydrochloride, such as the polymers identified by the INCI name Hydroxypropyl Guar Hydroxypropyltrimonium Chloride, which are available on the market, for example, under the name Jaguar C-162, made by Rhodia.

Other examples reported with their INCI names are quaternium-18 bentonite, quaternium-18/benzalkonium bentonite, quaternium M-18 hectorite and TEA-hydrochloride.

According to a preferred aspect of the invention, Hydroxypropyl Guar Hydroxypropyltrimonium Chloride, available on the market with the name Jaguar C-162, made by Rhodia, is used.

The non-ionic viscosity-controlling agents which may be used have the INCI names SPEG-180/Laureth-50 TMMG Copolymer, Butylene Glycol and Polyether-1, and are available from Rockwood under the trade name Pure Thix.

The non-ionic viscosity-controlling agents may also be polysaccharides, cellulose, methylcellulose, ethylcellulose, hydroxyethylcellulose, methylhydroxyethylcellulose and methylhydroxypropylcellulose.

The polyurethane viscosity-controlling agents usable may be polymers with a polyurethane base such polyurethane-30 (LuvigelSTAR® made by BASF). EO/PO block copolymers, such as Pluronics® made by BASF, may also be used. Other urethane viscosity-controlling agents called Dermothix,™ available from Alzo International Inc., may be also be included in the composition according to the invention. PEG-100 Stearyl Ether Dimer IPDI or PEG-75 Stearyl Ether Dimer IPDI (reported with their INCI names), commercially available under the name of Dermothix, may preferably be used.

The composition according to the invention may also contain polysaccharide and heteropolysaccharide viscosity-controlling agents, such as starches and derivatives thereof, cellulose and derivatives thereof, xanthan gum and carrageenans. The preferred heteropolysaccharides include xanthan gums such as Keltrol®T by Kelco and Natrosol®250HHR by Ashland. The preferred starch has the INCI name Hydroxypropyl Starch Phosphate, such as Structure® XL by National Starch.

Other additional ingredients of the cosmetic composition according to the invention include heat-sensitive polymers such as bis-methoxy PEG-13 PEG-438/PPG-110 SMDI copolymer (ExpertGel® EG 56), bis-methoxy PEG-13 PEG-502/PPG-57/SMDI copolymer (ExpertGel® EG 230), or a mixture thereof; said copolymers are available from PolymerExpert.

The cosmetic composition according to the invention may also include solvents, fats, polymers, oligosaccharides and modified oligosaccharides, carbohydrates and derivatives, polyols and derivatives, triglycerides, hydrocarbons, lanolin and derivatives, opacifiers, silicones, hydrolysed proteins, amino acids, complexing agents, UV filters, pigments, preservatives and fragrances.

The water-soluble organic solvents usable may be glycols, glycol ethers and polyols containing 2 to 6 carbon atoms. The glycols may be ethylene, propanediol or butanediol glycols. For example, the glycol ethers usable (reported with their INCI names) are ethylglycol, ethyldiglycol, diethyleneglycol, triethyleneglycol and dipropyleneglycol. The polyols usable (reported with their INCI names) include glycerol, trimethylolpropane and diglycerol. Examples of alkyl carbonates include (reported with their INCI names) ethylene carbonate and propylene carbonate.

Polyalkyl glycols are, for example, polyethylene glycols, polypropylene glycols, and related products to which ethylene oxide with a molecular weight of up to 1000 D is added.

The organic solvent may constitute up to 30% by weight of the total weight of the composition.

The fats may be fatty alcohols containing 10 to 30 carbon atoms, such as the following (reported with their INCI names): Capric Alcohol, Lauryl Alcohol, Myristyl Alcohol, Cetyl Alcohol, Stearyl Alcohol and Behenyl Alcohol. Lauryl Alcohol or Myristyl Alcohol is preferably usable.

The fats may constitute 0.5% to 20% by weight of the total weight of the composition, preferably from about 1% to 15%.

The composition according to the invention may contain cationic polymers called, with the INCI nomenclature, polyquaternium, vinylpyrrolidone/N-vinylimidazole salt copolymers (available under the trade name “Luviquat®”), N-vinylpyrrolidone/dimethyl-aminoethylmethacrylate copolymers, quaternised with diethyl sulphate, N-vinyl caprolactam/N-vinylpyrrolidone/N-vinylimidazoline copolymer salts; cationic cellulose derivatives (Polyquaternium-4 and -10), acrylamide/diallyldimethylammonium hydrochloride copolymer (Polyquaternium-7). A particular cationic polymer is poly(methacryloyloxyethyltrimethylammonium hydrochloride), with the INCI name of Polyquaternium-37.

Cationic polymers may constitute 0.1% to 5% by weight of the total weight of the composition, preferably from about 0.1% to 1%.

The composition may also contain oligosaccharides and modified oligosaccharides; of the latter, Oligoquat®M, which has the INCI name Stearyl Dihydroxypropyldimonium Oligosaccharides, and is available from the company Lonza, is preferred.

Oligosaccharides and modified oligosaccharides may constitute about 0.1% to 10% of the total weight of the composition, preferably from about 0.2% to 5%.

The composition according to the invention may also contain sugars, polyols and monosaccharide or disaccharide derivatives.

Said compounds may constitute about 0.1% to 10% of the total weight of the composition, preferably from about 0.2% to 5%.

The composition may also contain silicones (dimethicones, phenylpolysiloxanes, cyclomethicones, etc.) to obtain given properties. The usable compounds include the product sold by Dow Corning with the name of Dow Corning® (DC) 556 Cosmetic Fluid (INCI name: Phenyl Trimethicone), DC 190 (INCI name PEG/PPG-18/18 Dimethicone), DC 193 (INCI name PEG-12 Dimethicone), DC 200, DC 1401 (INCI name Cyclomethicone, Dimethiconol) and DC 1403 (INCI name Dimethicone, Dimethiconol), and the commercial product DC 244, DC 344 and DC 345 (INCI name for each one: Cyclomethicone) by Dow Corning, Q2-7224 (manufacturer: Dow Corning); a stabilised trimethylsilylamodimethicone, Dow Corning 929 emulsion (contains a modified hydroxyamino silicone also called amodimethicone), SM-2059 (manufacturer: General Electric), SLM-55067 (manufacturer: Wacker), and AbilQuat 3270 and 3272 (manufacturer: Th. Goldschmidt; dipolydimethylsiloxanediquaternarium, INCI name: Quaternium-80).

The silicones are preferably present in quantities ranging from 0.1% to 5% by weight of the total weight of the composition, more preferably from 0.3% to 2.5%.

The composition according to the invention may also include triglycerides; they may be animal, but preferably vegetable products such as almond oil, argan oil, avocado oil, calophyllum oil, castor oil, sesame oil, olive oil, jojoba oil, babassu oil, shea butter, linseed oil or sunflower oil.

The triglycerides are preferably present in quantities ranging from 0.1% to 30% by weight of the total weight of the composition, more preferably from 0.1% to 10%.

The composition according to the invention may also include hydrocarbons, which may be paraffins, isoparaffins, cycloparaffins, terpenes and terpenoids, such as mineral oil, vaseline, microcrystalline wax, ceresin, ozocherite, isohexadecane, diisooctylcyclohexane, squalene and squalane.

The hydrocarbons are preferably present in a quantity ranging from 0.1% to 30% by weight of the total weight of the composition, more preferably from 0.1% to 10%.

According to one aspect of the invention, lanolin and derivatives thereof may be present in the composition in quantities ranging from 0.1% to 30% by weight of the total weight of the composition, more preferably from 0.1% to 10%.

The composition may also contain opacifiers selected from the compounds listed herein by their INCI name: ammoniumstyrene/acrylatescopolymer, DEA-styrene/acrylates/DVB copolymer, guanine, Mica, styrene/acrylamide copolymer, styrene/acrylates copolymer, styrene/DVB copolymer; opacifiers having rheological corrector properties such as behenamide, erucamide, Nylon-12, Nylon-66, oleamide, oleylpalmitamide, stearamide, stearamide DEA-distearate, stearamide DIBA-distearate and stearylerucamide.

Said ingredients may be present in quantities ranging from 0.1% to 3% by weight of the total weight of the composition, preferably from 0.1% to 1%.

The composition according to the invention may also contain hydrolysed proteins of animal origin, such as hydrolysed proteins from elastin, keratin, silk and milk, which may also be in the form of a salt. Some products are available on the market, for example, with the trade name of Dehylan® (Cognis), Promois® (SeiwaKasei Co. Ltd.), Collapuron® (Cognis), Nutrilan® (Cognis), Gelita-Sol® (Deutsche Gelatine FabrikenStoess& Co), Lexein® (Inolex) and Kerasol® (Croda).

According to the invention, the proteins or hydrolysed proteins may also be of plant origin, such as proteins or hydrolysed proteins obtained from soya, almonds, peas, potatoes, flax, corn or wheat. Some products are available on the market under the trade name of Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

The hydrolysed products may be derivatives, such as Keramimic 2.0® by Croda.

The hydrolysed proteins or derivatives thereof may preferably be present in the composition in quantities ranging from 0.1 to 10% by weight of the total weight of the composition, preferably from 0.1 to 2.5%.

According to a further aspect of the invention, the composition may include the following amino acids: glycine, sarcosine, lysine, serine, glucosamine, glutamic acid, carnitine, acetylcarnitine, alanine, arginine, asparagine, aspartic acid, cysteine, glutamine, histidine, isoleucine, leucine, lysine, methionine, phenylalanine, proline, serine, threonine, tryptophan, tyrosine and valine. The preferred amino acids are arginine, asparagine, glutamine, histidine, lysine, proline and tryptophan.

The amino acids may be present in the composition in quantities ranging from 0.01 to 10% by weight of the total weight of the composition, preferably from 0.01 to 2.5%.

The composition may also include complexing agents, which may be selected from chelating agents, sequestering agents and the corresponding salts. Examples of chelating agents include ethylenediaminetetraacetic acid (EDTA), nitrilotriacetic acid, ethyleneglycol-bis(β-amino-ethyl ether)-N,N-tetraacetic acid and ethylenediamine N,N′-disuccinic acid (EDDS).

The chelating or sequestering agents may be present in the composition in quantities ranging from 0.05% to 10% by weight of the total weight of the composition, preferably from 0.05% to 2%.

According to the invention, sunscreens may be added to the composition in quantities ranging from 0.01% to 10% by weight of the total weight of the composition.

The cosmetic composition may also include at least one direct hair dye; said direct dyes may be non-ionic, cationic or anionic.

The non-ionic dyes usable may be selected, for example, from the following (INCI name in brackets): 2-amino-3-nitrophenol; 2-[(2-hydroxyethyl)amino]-1-methoxy-5-nitrobenzene; 1-(2-hydroxyethoxy)-3-methylamino-4-nitrobenzene; 2,3-(dihydroxypropoxy)-3-methylamino-4-nitrobenzene; 1-[(2-ureidoethyl)amino]-4-nitrobenzene; 4-[(2-hydroxyethyl)amino]-3-nitro-1-methylbenzene; 1-[(2-hydroxyethyl)amino]-2-nitrobenzene (HC Yellow No. 2); 1-(2-hydroxyethoxy)-2-[(2-hydroxyethyl)amino]-5-nitrobenzene (HC Yellow No. 4); 1-amino-2-[(2-hydroxyethyl)amino-5-nitrobenzene (HC Yellow No. 5); 4-[(2,3-dihydroxypropyl)amino]-3-nitro-1-trifluoromethylbenzene (HC Yellow No. 6); 1-(4-aminophenylazo)-2-methyl-4-(bis-2-hydroxyethyl)aminobenzene (HC Yellow No. 7); 3-[(2-aminoethyl)amino]-1-methoxy-4-nitrobenzene hydrochloride (HC Yellow No. 9); 1-chloro-2,4-bis-[(2-hydroxyethyl)amino]-5-nitrobenzene (HC Yellow No. 10); 2-[(2-hydroxyethyl)amino]-5-nitrophenol (HC Yellow No. 11); 1-chloro-4-[(2-hydroxyethyl)amino]-3-nitrobenzene (HC Yellow No. 12); 4-[(2-hydroxyethyl)amino]-3-nitro-1-trifluoromethyl-benzene (HC Yellow No. 13); 4-[(2-hydroxyethyl)amino]-3-nitro-benzonitrile (HC Yellow No. 14); 4-[(2-hydroxyethyl)amino]-3-nitro-benzamide (HC Yellow No. 15); 1,4-diamino-2-nitrobenzene; 1,4-bis[(2-hydroxyethyl)amino]-2-nitrobenzene; 2-amino-4,6-dinitro-phenol; 4-amino-3-nitrophenol; 1-amino-5-chloro-4-[(2-hydroxyethyl)amino-2-nitrobenzene; 4-[(2-hydroxyethyl)amino]-3-nitrophenol (trade name Jarocol NHEAP); 4-[(2-nitrophenyl)amino]phenol (HC Orange No. 1); 1-[(2-aminoethyl)amino]-4-(2-hydroxyethoxy)-2-nitrobenzene (HC Orange No. 2); 4-(2,3-dihydroxypropoxy)-1-[(2-hydroxyethyl)amino]-2-nitrobenzene (HC Orange No. 3); 2-[(2-hydroxyethyl)amino]-4,6-dinitro-phenol; 4-ethylamino-3-nitrobenzoic acid; 2-[(4-amino-2-nitrophenyl)amino]-benzoic acid; 2-chloro-6-ethylamino-4-nitrophenol; 2-amino-6-chloro-4-nitrophenol; 4-Nitro-o-phenylenediamine; 4-[(3-hydroxypropyl)amino]-3-nitrophenol; 2,5-diamino-6-nitropyridine; 1,2,3,4-tetrahydro-6-nitro-quinoxaline; 4-amino-2-nitro-diphenylamine (HC Red No. 1); 4-amino-1-[(2-hydroxyethyl)amino-2-nitrobenzene (HC Red No. 3); 1-amino-4-[(2-hydroxyethyl)amino-2-nitrobenzene (HC Red No. 7); 1-amino-5-chloro-4-[(2,3-dihydroxypropyl)amino]-2-nitrobenzene (HC Red No. 10); 5-chloro-1,4-[di(2,3-dihydroxypropyl)amino]-2-nitrobenzene (HC Red No. 11); 1-amino-4-[di(2-hydroxyethyl)amino]-2-nitrobenzene hydrochloride (HC Red No. 13); 7-amino-3,4-dihydro-6-nitro-2H-1,4-benzoxazine (HC Red No. 14); 4-[(3-hydroxypropyl)amino]3nitrophenol (HC Red BN); 1-amino-3-methyl-4-[(2-hydroxyethyl)amino]-6-nitrobenzene (HC Violet No. 1); 1-(3-hydroxypropylamino)-4-[di(2-hydroxyethyl)amino]-2-nitrobenzene (HC Violet No. 2); 1-(2-hydroxyethyl)amino-2-nitro-4-[di(2-hydroxyethyl)amino]-benzene (HC Blue No. 2); 1-methylamino-4-[methyl-(2,3-dihydroxypropyl)amino]-2-nitrobenzene (HC Blue No. 6); 1-[(2,3-dihydroxypropyl)amino]-4-[ethyl-(2-hydroxyethyl)amino-2-nitrobenzene hydrochloride (HC Blue No. 9); 1-[(2,3-dihydroxypropyl)amino]-4-[methyl-(2-hydroxyethyl)amino-2-nitrobenzene (HC Blue No. 10); 4-[di(2-hydroxyethyl)amino]-1-[(2-methoxyethyl)amino-2-nitrobenzene (HC Blue No. 11); 4-[ethyl-(2-hydroxyethyl)amino]-1-[(2-hydroxyethyl)amino-2-nitrobenzene hydrochloride (HC Blue No. 12); 2-((4-amino-2-nitrophenyl)amino)-5-dimethylaminobenzoic acid (HC Blue No. 13); N,N′-bis(2-hydroxyethyl)-2-nitro-p-phenylenediamine; 1,4-bis[(2,3-dihydroxypropyl)amino]-9,10-anthracenedione (HC Blue No. 14); 1-amino-5-chloro-4-(2,3-dihydroxypropyl)amino-2-nitrobenzene (HC Red No. 10); 1-chloro-2,5-di[(2,3-dihydroxypropyl)amino]-4-nitrobenzene (HC Red No. 11); Isatin.

Non-ionic disperse dyes may also be used, such as 1,4-di[(2,3-dihydroxypropyl)amino]-9,10-anthracenedione, 2-[(2-amino ethyl)amino-9,10-anthracenedione, 1-[(3-aminopropyl)amino-9,10-anthracenedione, 1,4-diamino-2-methoxy-9,10-anthracenedione (C.I. 62015, Disperse Red 11); 1-amino-4-hydroxy-9,10-anthracenedione (C.I. 60710, Disperse Red 15); 1,4-diamino-5-nitro-9,10-anthracenedione (C.I. 62030, Disperse Violet 8); 1,4-bis[(2-hydroxyethyl)amino]-9,10-anthracenedione (C.I. 61545; Disperse Blue 23); 1-[(2-hydroxyethyl)amino]-4-[(3-hydroxypropyl)amino-9,10-anthracenedione; 1,4-bis[(3-hydroxypropyl)amino]-9,10-anthracenedione; 2-((4-(acetylamino)phenyl)azo)-4-methylphenol (C.I. 11855; Disperse Yellow 3); 1-(4′-aminophenylazo)-4-nitrobenzene (C.I. 11005; Disperse Orange 3); Disperse Black 9, Disperse Red 17, Disperse Violet 1, Disperse Violet 4, Disperse Violet 15, Disperse Violet 27, Disperse Blue 1, Disperse Blue 3, Disperse Blue 7, Disperse Blue 72, and Disperse Blue 377, 2-hydroxyethyl picramic acid, 4-nitrophenyl aminourea, 3-methylamino-4-nitrophenoxyethanol and 2-nitro-5-glycerylmethylaniline.

The preferred non-ionic dyes are HC Yellow No. 7, 2-amino-6-chloro-4-nitrophenol, 4-amino-3-nitrophenol, HC Orange No. 1, HC Red No. 1, HC Red No. 3, HC Red No. 13, Disperse Red 17, HC Blue No. 2, 4-[(2-hydroxyethyl)amino]-3-nitrophenol (trade name Jarocol NHEAP); 4-[(3-hydroxypropyl)amino]-3-nitrophenol (trade name: Jarocol Red BN), 4-[ethyl-(2-hydroxyethyl)amino]-1-[(2-hydroxyethyl)amino]-2-nitrobenzene hydrochloride (HC Blue No. 12), 1-(2-hydroxyethoxy)-2-[(2-hydroxyethyl)amino]-5-nitrobenzene (HC Yellow No. 4) and 1-[(2-hydroxyethyl)amino]-2-nitrobenzene (HC Yellow No. 2).

The cationic dyes usable may be selected, for example, from the following (reported with the INCI name): Basic Yellow 57, Basic Yellow 87, Basic Brown 16, Basic Brown 17, Basic Orange 31, Basic Orange 69, Basic Red 51, Basic Red 76, Basic Blue 124, Hydroxyanthraquinoneaminopropylmethylmorpholiniummethosulfate and HC Blue 15, HC Blue 16 and HC Blue 17, Basic Blue 99; in addition, they may be selected from: [2-((2,4-dimethoxyphenyl)amino)ethenyl]-1,3,3-trimethyl-3H-indol-1-ium chloride (C.I. 48055; Basic Yellow 11); bis[4-(dimethylamino)phenyl][4-(methylamino)phenyl]carbenium-chloride (C.I. 42535; Basic Violet 1); Benzenamine, (4-amino-3-methylphenyl)(4-imino-3-methyl-2,5-cyclohexadien-1-ylidene)methyl]-2-methyl-,monohydrochloride (C.I. 42520 Basic Violet 2); tris[4-(dimethylamino)phenyl]carbenium-chloride (C.I. 42555; Basic violet 3); 2-[3,6-(diethylamino)dibenzopyranium-9-yl]-benzoic acid chloride (C.I. 45170; Basic Violet 10); di(4-aminophenyl)(4-amino-3-methylphenyl)carbenium-chloride (C.I. 42510; Basic Violet 14); 3-[(E)-(3-chloro-4-hydroxyphenyl)diazenyl]-2,1-benzisothiazole-5-sulfonamide (HC Blue 18), also the colorants cited in WO 2014/202150 are incorporated by reference. WO 2014/202150 in its entirety, in particular from page 10 line 13 to page 15, line 22; from page 15 line 27 to page 16 line 24; from page 17 line 1 to page 18 line 16.

The preferred cationic dyes are Basic Yellow 57, Basic Yellow 87, Basic Brown 16, Basic Brown 17, Basic Orange 31, Basic Red 51, Basic Red 76, HC Blue 15, HC Blue 16, Basic Blue 75, Basic Blue 99 and Basic Blue 124.

The anionic dyes usable may be selected, for example, from the following (reported here with the INCI/CTFA name (INCI—International Nomenclature of Cosmetic Ingredients, Decision of the European Community 96/335/EC as amended by 2006/257/EC; CTFA—Cosmetic Ingredient Dictionary Handbook, by the Personal Care Products Council, Washington, USA 15th ed., 2014); or the corresponding Color Index): Acid Black 1 (CI 20470); Acid Blue 1 (CI 42045); Food Blue 5 (CI 42051); Acid Blue 3 (CI 42051); Acid Blue 7 (CI 42080); Acid Blue 9 (CI 42090); Acid Blue 74 (CI 73015); Acid Red 18 (CI 16255); Acid Red 27 (CI 16185); Acid Red 33 (CI 17200); Acid Red 35 (CI 18065); Acid Red 40 (CI 18070); Acid Red 52 (CI 45100); Acid Red 87 (CI 45380); Acid Red 92 (CI 45410); Acid Orange 7 (CI 15510); Acid Violet 43 (CI 60730); Acid Yellow 1 (CI 10316); Acid Yellow 3 (CI 47005); Acid Yellow 23 (CI 19140); Acid Yellow 36 (CI13065) Food Yellow 8 (CI 14270); Acid Orange 24 (CI20170); Acid Yellow 9 (CI 13015); Acid Violet 49 (CI 42640); Acid Green 25, D&C Black No. 2; D&C Black No. 3; FD&C Blue No. 1; D&C Blue No. 4; D&C Brown No. 1; FD&C Green No. 3; D&C Green No. 6; D&C Green No. 8; D&C Orange No. 4; D&C Orange No. 5; D&C Orange No. 10; D&C Orange No. 11; FD&C Red No. 4; D&C Red No. 6; D&C Red No. 7; D&C Red No. 17; D&C Red No. 21; D&C Red No. 27; D&C Red No. 30; D&C Red No. 31; D&C Red No. 34; D&C Red No. 36; FD&C Red No. 40; Ext. D&C Violet No. 2; FD&C Yellow No. 6; D&C Yellow No. 7; D&C Yellow No. 8; D&C Yellow No. 11.

The preferred anionic dyes are Acid Yellow 1, Acid Yellow 3, Acid Yellow 23, Acid Orange 7, Acid Red 33, Acid Red 40, Acid Red 52, Acid Red 92, Acid Violet 43, Acid Blue 7, Acid Blue 9 and Acid Blue 62.

Said non-ionic, cationic and anionic dyes may be contained in the composition alone or in mixtures, in quantities ranging from about 0.01 to 4.0% by weight of the total weight of the composition.

The composition may also include natural direct dyes, such as those based on lawsone, juglone, alizarine, purpurine, carminic acid, kermesic acid, purpurogallin, protocatechualdehyde, indigo, isatin, curcumin, spinulosine and apigenidine. Extracts or decoctions containing said natural dyes may also be used.

For further typical colouring compounds which may be used in the composition, reference may expressly be made to the “Dermatology” series, edited by Ch. Culnan and H. Maibach, Verlag Marcel Dekker Inc., New York, Basel, 1986, volume 7, Ch. Zviak; “The Science of Hair Care”, chapter 7, pp. 248-250 (substantive dyes), F. Mearelli “Le piante tintorie nella colorazione dei capelli”, and the “European Inventory of Cosmetic Raw Materials”, published by the European Union, obtainable on disc from BundesverbandDeutscher Industrie-und HandelsunternehmenfurArzneimittel, Reformwaren und Körperpflegemittel e.V., Mannheim.

Pigments, such as iron oxides, titanium oxides, zinc oxides, chromium oxides, ultramarine, manganese violet and ferric ferrocyanide may also be used according to the invention. Other particular pigments which may be used are those marketed under the name WATERSPERSE® by the company S.A. COLOR; UNIPURE by the company SENSIENT; CELLINI® by the company BASF; DISTINCTIVE® by the company RESOURCE OF NATURE, COLORONA® by the company MERK, and WD by the company DAITO KASEI.

Said pigments may be included in the composition in quantities ranging from 0.01 to 10% by weight of the total weight of the composition, preferably from 3 to 8%.

The cosmetic composition may also include preservatives and fragrances in quantities ranging from 0.01 to 2% by weight of the total weight of the composition.

The pH of the composition may range between 0.5 and 3.0, preferably between 0.8 and 2.8, and more preferably between 0.8 and 2.0.

According to the invention, pH correctors may be added in quantities ranging from 0.01 to 10%, preferably from 0.01 to 2%, by weight of the total weight of the composition.

The viscosity of the composition before shaking may range between 200 cP and 3000 cP, preferably between 500 cP and 1800 cP, measured with a Brookfield RVII instrument with spindle 3 at the speed of 10 rpm at the temperature of 25° C.

The ready-for-use cosmetic composition according to the invention is designed for use with a device able to form a foam at the time of shaking which has a ratio between height and largest diameter of hole lower than (approximately) 3:1, and a ratio between the volume of the device and the composition it contains ranging between 0.08 and 0.25, expressed as volume/volume ratio, preferably 0.16 v/v.

Said device may have a volume ranging from 100 to 1000 ml, preferably from 200 to 500 ml.

The device may have a cap of various shapes able to close the hole during shaking.

The device illustrated and described in WO 2013/057129 and US 2012/0305416 A1 may preferably be used.

The invention also relates to a hair reshaping or hair straightening method comprising the use of the cosmetic composition according to the invention and the above-mentioned device, as follows:

(1) mix glyoxylic acid and/or at least one derivative of formula (I), (II) or (III) as defined above, at least one foaming agent selected from anionic, cationic and non-ionic surfactants, and at least one viscosity-controlling agent, to obtain a composition having a viscosity ranging between 200 and 3000 cP, and shake in a device having a ratio between outlet hole height and diameter of less than 3:1 for 5 to 60 seconds, preferably 10 to 30 seconds, until a foam is obtained;

(2) pick up the resulting foam with the hands, apply it to the hair, and leave to process for 5 to 45 minutes;

(3) reshape/straighten hair using a heat source.

The hair should preferably be washed with shampoo and dried before coming into contact with the foam according to the invention.

The heat may be supplied by a hood dryer, hairdryer, straightening iron or other conventional equipment. Heat may be also supplied at point (2).

The hair may be rinsed and dried before or immediately after reshaping/straightening.

A post-treatment may be applied to the hair.

According to a preferred embodiment of the invention, a first method includes the following steps:

(i) wash hair with shampoo and dry;

(ii) mix glyoxylic acid and/or at least one derivative of formula (I), (II) or (III) as defined above, at least one foaming agent selected from anionic, cationic and non-ionic surfactants, and at least one viscosity-controlling agent, to obtain a composition having a viscosity ranging between 200 and 3000 cP, and shake in the device having a ratio between outlet hole height and diameter of less than 3:1 for 10 to 30 seconds, preferably until the liquid is no longer heard to move in the container of the device, until a foam is obtained;

(iii) pick up the foam with the hands, apply it to the hair, leave to process for 5 to 45 minutes, then comb and dry, preferably with a hairdryer;

(iv) reshape/straighten hair with heat and reshaping equipment;

(v) rinse hair and dry;

(vi) optionally treat hair with a post-treatment.

According to a further preferred embodiment of the invention, a second method includes the following steps:

(i) wash hair with shampoo and dry;

(ii) mix glyoxylic acid and/or at least one derivative of formula (I), (II) or (III) as defined above, at least one foaming agent selected from anionic, cationic and non-ionic surfactants, and at least one viscosity-controlling agent, to obtain a composition having a viscosity ranging between 200 and 3000 cP, and shake in the device having a ratio between outlet hole height and diameter of less than 3:1 for 10 to 30 seconds, preferably until the liquid is no longer heard to move in the container of the device, until a foam is obtained;

(iii-a) pick up the foam with the hands, apply it to the hair, and leave to process for 5 to 45 minutes;

(iii-b) a heat source other than a hairdryer (such as a hood) may optionally be used during step (iii-a);

(v-a) rinse hair with tap water;

(vi) optionally treat hair with a post-treatment;

(iv) reshape/straighten hair with heat and reshaping/straightening equipment.

According to a preferred embodiment of the invention, the composition applied to the hair is left to process for approximately 5 to 45 minutes, preferably 15 to 45 minutes, at room temperature or under a heat source, and then rinsed and dried with a hairdryer, thus preventing the development of fumes but ensuring the performance of the product. The rinsed and optionally dried hair may then be treated with a straightening iron for 5 to 10 consecutive cycles at a temperature exceeding 100° C., preferably between 150° C. and 250° C., and more preferably between 180° C. and 220° C.

The present invention quickly and easily creates a ready-to-use foam that maintains its characteristics unchanged, eliminating dripping throughout the application and processing time. Moreover, due to the absence of metal parts in the device, problems of corrosion due to the type of composition are avoided.

The present composition makes it possible to use simple devices which may be reused; moreover, for the above-mentioned reasons, particularly cheap containers may be selected.

### EXAMPLES

The following examples further illustrate the invention.

The percentages indicated in the examples are expressed by weight in relation to the total weight of the composition.

### Example 1

| Ingredients (INCI) | % |
|---|---|
| Aqua (water) | q.s. for 100 |
| Glyoxylic acid | 15 |
| Propylene glycol | 2 |
| Cocamidopropyl betaine | 5 |
| Cetrimonium chloride | 2 |
| Xanthan gum | 0.5 |

A device with the following dimensions was used:

Hole diameter: 8 cm

Height: 15 cm

The viscosity obtained is 1530 cP with probe 3 at 10 rpm measured with a Brookfield RVII instrument at the temperature of 25° C.

The pH of the composition is 1.0

### Example 2—Comparison Between the Foam According to the Invention and a Foam Produced with a Pump-Driven Foamer

Formulas E2A and E2B are set out below.

E2A is a conventional formula for use with a pump-driven foamer, while E2B is the formula according to the invention for use in a device with the following dimensions:

Hole diameter: 7.5 cm

Height: 13 cm

| Ingredients (INCI) | E2A % | E2B % |
|---|---|---|
| Aqua (water) | q.s. for 100 | q.s. for 100 |
| Glyoxylic acid | 17 | 17 |
| Propylene glycol | 5 | 5 |
| Cetrimonium chloride | 0.5 | 0.5 |
| PEG-15 cocopolyamine | 0.5 | |
| Parfum (fragrance) | 0.3 | 0.3 |
| Hydrolyzed keratin | 0.5 | 0.5 |
| PPG-1-PEG-9 lauryl glycol ether | 0.24 | |
| Trametes versicolor extract | 0.2 | 0.2 |
| PEG-40 hydrogenated castor oil | 0.16 | 0.16 |
| Disodium EDTA | 0.1 | 0.1 |
| Linum usitatissimum seed oil | 0.2 | 0.2 |
| Xanthan gum | | 0.4 |
| Cocamidopropyl betaine | | 4.2 |

viscosity of E2A: 15 cP with probe 1 and speed of 50 rpm

viscosity of E2B: 1450 cP with probe 3 and speed of 10 rpm

pH of E2A: 1.1

pH of E2B: 1.05

To compare the ability to prevent foam dripping in the laboratory, a support was positioned at a 30 degree angle. 1 g of each of the two foams generated by the two formulas E2A and E2B was applied to the end of the support. The two foams were left for 1 minute, and the distance traveled by the product due to the inclined plane was measured after 10, 20, 30 and 60 seconds (s). The data are set out in Table 1 in centimetres (cm) in relation to the time in seconds (s); as will be seen, the foam generated by the formula according to the invention (E2B) travels a much shorter distance than Formula E2A generated by the pump-driven foamer.

TABLE 1

| Formula | 10 s | 20 s | 30 s | 60 s |
|---|---|---|---|---|
| E2A | 5 cm | 11 cm | 14 cm | 17 cm |
| E2B | 0 cm | 0.5 cm | 0.5 cm | 1 cm |

A subjective value was also allocated to evaluate and verify the quality of the foam at time 0, and 1 minute, 5 minutes and 30 minutes after the time when it was obtained with the different devices, on the following value scale:

1—Completely liquefied

2—Loss of texture without liquefying

3—Slightly collapsed foam

4—Compact foam.

The results are set out in Table 2, which demonstrates that the foam generated by the formula according to the invention maintains a higher quality over time than the conventional foam.

TABLE 2

| Formula | 0 min | 1 min | 5 min | 30 min |
|---|---|---|---|---|
| E2A | 4 | 2 | 1 | 1 |
| E2B | 4 | 4 | 4 | 3 |

When compositions E2A and E2B were applied to models with curly hair (Model A), wavy hair (Model B) and slightly wavy hair (Model C), the following results in terms of reshaping effect were obtained:

TABLE 3

| Formula | Straightening | Dripping |
|---|---|---|
| Model A | | |
| E2A | 3 | 2 |
| E2B | 3 | 3 |
| Model B | | |
| E2A | 3 | 1 |
| E2B | 3 | 3 |
| Model C | | |
| E2A | 3 | 1 |
| E2B | 3 | 3 |

Table 4 shows the scales used to evaluate straightening and dripping.

TABLE 4
Straightening

| | |
|---|---|
| 1 | reshaping objective not achieved |
| 22 | reshaping objective not fully achieved |
| 33 | reshaping objective achieved: |

Dripping

| | |
|---|---|
| 11 | Dripping |
| 22 | Slight dripping |
| 33 | No dripping |

The following method was used to treat the hair of Models A and B:

(i) wash hair with shampoo and dry;

(ii) shake the composition of formula E2A in the device for 15 seconds and dispense the foam of formula E2A through the pump-driven foamer; in the case of Formula E2B, mix;

(iii) apply foam to hair, leave to process for 20 minutes, comb and dry with hairdryer;

(iv) reshape hair with a straightening iron at 200° C.; (v and vi) rinse hair and apply post-treatment;

(vii) rinse and dry.

The following method was used to treat the hair of Model C:

(i) wash hair with shampoo and dry;

(ii) shake the composition of formula E2A in the device for 20 seconds and dispense the foam of formula E2A through the pump-driven foamer; in the case of Formula E2B, mix;

(iii-a and iii-b) apply foam to hair and leave to process for 30 minutes, including 15 minutes under a heat source;

(v-a) rinse with water;

(vi) apply post-treatment;

(v-a) rinse with water;

(iv) reshape hair with a straightening iron at 200° C.

The conventional composition illustrated in example E2A was also tested in a vessel with the following dimensions:

Hole diameter: 7.5 cm

Height: 13 cm

After shaking, a foam was obtained that collapsed instantly into a liquid after 10 seconds, preventing the foam from being applied with the hands.

### Example 3

| Ingredients (INCI) | % |
|---|---|
| Aqua (water) | q.s. for 100 |
| Glyoxylic acid | 15 |
| Propylene glycol | 2 |
| Cocamidopropyl betaine | 5 |
| Cetrimonium chloride | 2 |
| Xanthan gum | 0.5 |
| HC Blue no. 15 | 0.01 |
| Basic Blue 124 | 0.1 |

Dark curly hair with a slight blue sheen was straightened with this formula.

The following method was used with a device having a hole diameter of 10 cm and a height of 15 cm:

(i) wash hair with shampoo and dry;

(ii) shake composition in the device for 15 seconds;

(iii) pick up foam and apply to hair, leave to process for 20 minutes, comb and dry with hairdryer.

### Example 4

| Ingredients (INCI) | % |
|---|---|
| Aqua (water) | q.s. for 100 |
| Glyoxylic acid | 15 |
| Propylene glycol | 2 |
| Cocamidopropyl betaine | 5 |
| Cetrimonium chloride | 2 |
| Xanthan gum | 0.5 |
| Citric acid | 0.05 |
| Sodium carbonate | 0.01 |
| Sorbitol | 0.05 |
| Ceteareth-25 | 0.01 |
| Red iron oxide | 0.5 |
| C.I. 77007 | 0.05 |

Dark curly hair with a slight red sheen was straightened with this formula.

The following method was used with a device having a hole diameter of 10 cm and a height of 15 cm:

(i) wash hair with shampoo and dry;

(ii) mix the composition in the device for 10 to 30 seconds, preferably until the liquid is no longer heard to move in the container of the device;

(iii-a) pick up the resulting foam with the hands, apply it to the hair, and leave to process for 5 to 45 minutes;

(iii-b) use hood as heat source during step (iii-a);

(v-a) rinse hair with tap water;

(vi) treat hair with a post-treatment;

(iv) reshape hair with heat and reshaping equipment.

### Example 5

Further examples of compositions according to the invention are reported below.

| Ingredients (INCI) | % | % | % | % | % | % | % | % | % | % |
|---|---|---|---|---|---|---|---|---|---|---|
| Aqua (water) | q.s. | q.s. | q.s. | q.s. for 100 | q.s. for 100 | q.s. for 100 | q.s. for 100 | q.s. for 100 | q.s. for 100 | q.s. for 100 |
| | for 100 | for 100 | for 100 | | | | | | | |
| Glyoxylic acid | 0 | 0 | 10 | 10 | 10 | 0 | 10 | 10 | 10 | 10 |
| Pyruvic acid | 21 | 0 | 10 | 0 | 10 | 10 | 10 | 10 | 10 | 10 |
| Glyoxyloyl | 0 | 25 | 10 | 10 | 0 | 10 | 10 | 10 | 10 | 10 |
| Carbocysteine | | | | | | | | | | |
| Propylene glycol | 2 | 2 | 2 | 2 | 2 | 2 | 2 | 2 | 2 | 2 |
| Cocamidopropyl | 5 | 5 | 5 | 5 | 5 | 5 | 5 | 5 | 5 | 5 |
| betaine | | | | | | | | | | |
| Cetrimonium | 2 | 2 | 2 | 2 | 2 | 2 | 2 | 2 | 2 | 2 |
| chloride | | | | | | | | | | |
| Xanthan gum | 0.5 | 0.5 | 0.5 | 0.5 | 0.5 | 0.5 | 0.5 | 0.5 | 0.5 | 0.5 |
| Citric acid | 0.05 | 0.05 | 0.05 | 0.05 | 0.05 | 0.05 | 0.05 | 0.05 | 0.05 | 0.05 |
| Sodium | 0.01 | 0.01 | 0.01 | 0.01 | 0.01 | 0.01 | 0.01 | 0.01 | 0.01 | 0.01 |
| carbonate | | | | | | | | | | |
| Sorbitol | 0.05 | 0.05 | 0.05 | 0.05 | 0.05 | 0.05 | 0.05 | 0.05 | 0.05 | 0.05 |
| Ceteareth-25 | 0.01 | 0.01 | 0.01 | 0.01 | 0.01 | 0.01 | 0.01 | 0.01 | 0.01 | 0.01 |
| Basic red 51 | 0 | 0 | 0 | 0 | 0 | 0 | 1 | 0 | 0 | 0 |
| Acid red 92 | 0 | 0 | 0 | 0 | 0 | 0 | 0 | 0.4 | 0 | 0 |
| HC Blue 15 | 0 | 0 | 0 | 0 | 0 | 0 | 0 | 0 | 0.2 | 0 |
| 2-AMINO-6- | 0 | 0 | 0 | 0 | 0 | 0 | 0 | 0 | 0 | 0.8 |
| CHLORO-4- | | | | | | | | | | |
| NITROPHENOL | | | | | | | | | | |

1. Cosmetic composition for semi-permanent hair straightening or semi-permanent hair reshaping comprising:
glyoxylic acid and/or at least one derivative of formula (I), (II) or (III): 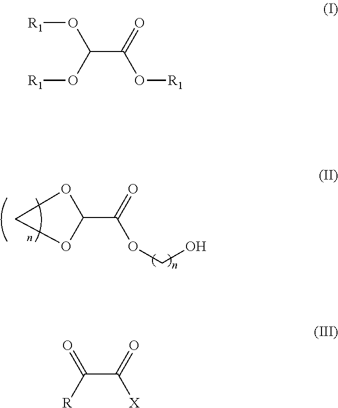
wherein:
in structural formula (I), R1 is selected from H, a C1-C8 alkyl group, a C1-C8 monohydroxyalkyl group, a C3 or C4 dihydroxyalkyl group;
in structural formula (II), n is 2 or 3;
in structural formula (III), R is H or —CH₃ and X is —OH or —NHR₂ wherein R₂ is an amino-acid radical and N is the amino group corresponding to the amino-acid group;
ranging from 5% to 25% by weight of the total weight of the composition,
at least one foaming agent selected from anionic, cationic, nonionic surfactants, and
at least one viscosity-controlling agent to obtain a viscosity ranging from 200 to 3000 cP,
in the form of a foam.

2. Cosmetic composition in the form of a foam obtainable by a process comprising the following steps:
a) mixing:
(1) glyoxylic acid and/or at least one derivative of formula (I), (II) or (III): 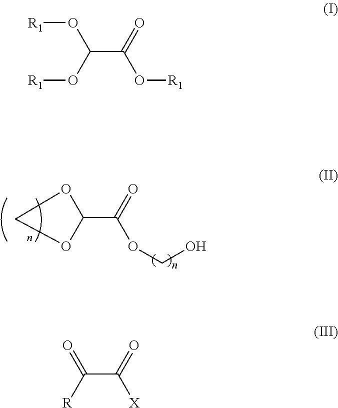
wherein:
in structural formula (I), R1 is selected from H, a C1-C8 alkyl group, a C1-C8 monohydroxyalkyl group, a C3 or C4 dihydroxyalkyl group;
in structural formula (II), n is 2 or 3;
in structural formula (III), R is H or —CH₃ and X is —OH or —NHR₂ wherein R₂ is an amino-acid radical and N is the amino group corresponding to the amino-acid group;
(2) at least one foaming agent selected from anionic, cationic, and nonionic surfactants, and
(3) at least one viscosity-controlling agent,
to obtain a composition having a viscosity ranging from 200 to 3000 cP;
b) shaking the composition obtained in step a) in a device having a ratio between outlet hole height and diameter lower than 3:1.

3. Cosmetic composition according to claim 1, wherein in structural formula (III) R is H and X is —OH or —NHR₂ wherein R₂ is an amino-acid radical and N is the amino group corresponding to the amino-acid group.

4. Cosmetic composition according to claim 1, wherein the viscosity of the composition ranges from 500 to 1800 cP.

5. Cosmetic composition according to claim 2, wherein the ratio between the volume of the device and the composition of step a) contained therein ranges from 0.08 to 0.25 volume/volume.

6. (canceled)

7. Cosmetic composition according to claim 1, having a pH ranging from 0.5 to 3.0.

8. Cosmetic composition according to claim 7, further comprising at least one nonionic, cationic or anionic direct hair dye or a pigment.

9. Cosmetic composition according to claim 8, wherein at least one nonionic dye is selected from the group consisting of HC Yellow No. 7, 2-amino-6-chloro-4-nitrophenol, 4-amino-3nitrophenol, HC Orange No. 1, HC Red No. 1, HC Red No. 3, HC Red No. 13, Disperse Red 17, HC Blue No. 2, 4-[(2-hydroxyethyl)amino]-3-nitrophenol; 4-[(3-hydroxypropyl)amino]-3-nitrophenol, 4-[ethyl-(2-hydroxyethyl)amino]-1-[(2-hydroxyethyl)amino]-2-nitrobenzene hydrochloride, 1-(2-hydroxyethoxy)-2-[(2-hydroxyethyl)amino]-5-nitrobenzene and 1-[(2-hydroxyethyl)amino]-2-nitrobenzene.

10. Cosmetic composition according to claim 8, wherein at least one cationic dye is selected from the group consisting of Basic Yellow 57, Basic Yellow 87, Basic Brown 16, Basic Brown 17, Basic Orange 31, Basic Red 51, Basic Red 76, HC Blue 15, HC Blue 16, Basic Blue 75, Basic Blue 99 and Basic Blue 124.

11. Cosmetic composition according to claim 8, wherein at least one anionic dye is selected from the group consisting of Acid Yellow 1, Acid Yellow 3, Acid Yellow 23, Acid Orange 7, Acid Red 33, Acid Red 40, Acid Red 52, Acid Red 92, Acid Violet 43, Acid Blue 7, Acid Blue 9 and Acid Blue 62.

12. Cosmetic composition according to claim 8, wherein the at least one direct dye or pigment is present in the composition in an amount ranging from 0.01 to 10.0% by weight of the total composition weight.

13. (canceled)

14. Method for semi-permanent hair straightening or semi-permanent hair reshaping comprising the following steps:
(1) mixing:
glyoxylic acid and/or at least one derivative of formula (I), (II) or (III), 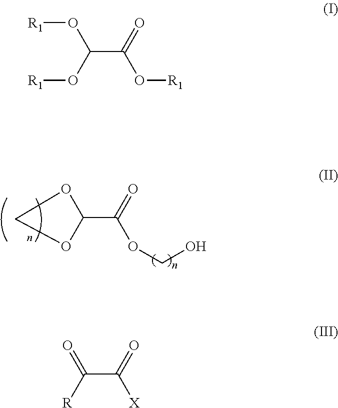
wherein:
in structural formula (I), R1 is selected from H, a C1-C8 alkyl group, a C1-C8 monohydroxyalkyl group, a C3 or C4 dihydroxyalkyl group;
in structural formula (II), n is 2 or 3;
in structural formula (III), R is H or —CH₃ and X is —OH or —NHR₂ wherein R₂ is an amino-acid radical and N is the amino group corresponding to the amino-acid group;
ranging from 0.5% to 30% by weight of the total weight of the composition,
at least one foaming agent selected from anionic, cationic, nonionic surfactants, and
at least one viscosity-controlling agent,
to obtain a composition having a viscosity ranging from 200 to 3000 cP according to claim 2 in a device having a ratio between outlet hole height and diameter lower than 3:1, for 5 to 60 seconds, to obtain a foam;
(2) picking up the resulting foam with the hands and applying it to the hair, and leaving it on the hair for 5 to 45 minutes;
(3) reshaping or straightening hair using a heat source.

15. Method for semi-permanent hair straightening or semi-permanent hair reshaping comprising the following steps:
(i) washing hair with shampoo and drying hair;
(ii) mixing:
glyoxylic acid and/or at least one derivative of formula (I), (II) or (III) 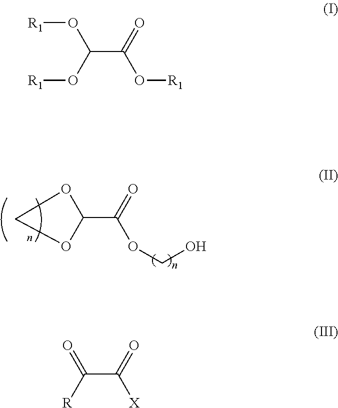
wherein:
in structural formula (I), R1 is selected from H, a C1-C8 alkyl group, a C1-C8 monohydroxyalkyl group, a C3 or C4 dihydroxyalkyl group;
in structural formula (II), n is 2 or 3;
in structural formula (III), R is H or —CH₃ and X is —OH or —NHR₂ wherein R₂ is an amino-acid radical and N is the amino group corresponding to the amino-acid group;
ranging from 0.5% to 30% by weight of the total weight of the composition,
at least one foaming agent selected from anionic, cationic, nonionic surfactants, and
at least one viscosity-controlling agent,
to obtain a composition having a viscosity ranging from 200 to 3000 cP according to claim 2 in a device having a ratio between outlet hole height and diameter lower than 3:1, for 5 to 60 seconds to obtain a foam;
(iii) picking up the foam according to the invention with the hands and applying it to hair, leaving it on the hair from 5 to 45 minutes, then combing and drying hair preferably with a hairdryer;
(iv) reshaping or straightening hair with heat and reshaping aids;
(v) rinsing and drying hair;
(vi) optionally treating hair with a post-treatment.

16. Method for semi-permanent hair straightening or semi-permanent hair reshaping comprising the following steps:
(i) washing hair with shampoo and drying hair;
(ii) mixing:
glyoxylic acid and/or at least one derivative of formula (I), (II) or (III) 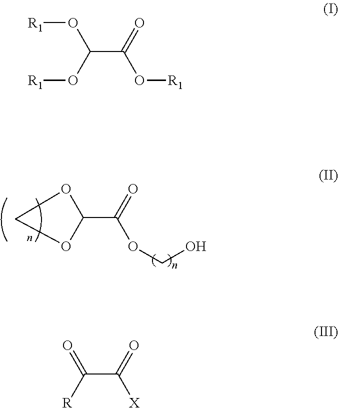
wherein:
in structural formula (I), R1 is selected from H, a C1-C8 alkyl group, a C1-C8 monohydroxyalkyl group, a C3 or C4 dihydroxyalkyl group;
in structural formula (II), n is 2 or 3;
in structural formula (III), R is H or —CH₃ and X is —OH or —NHR₂ wherein R₂ is an amino-acid radical and N is the amino group corresponding to the amino-acid group;
ranging from 0.5% to 30% by weight of the total weight of the composition,
at least one foaming agent selected from anionic, cationic, nonionic surfactants, and
at least one viscosity-controlling agent,
to obtain a composition having a viscosity ranging from 200 to 3000 cP according to claim 2 in a device having a ratio between outlet hole height and diameter lower than 3:1, for 5 to 60 seconds to obtain a foam;
(iii-a) picking up the foam with the hands and applying it to hair, and leaving it on the hair for 5 to 45 minutes;
(iii-b) optionally during step (iii-a) using hood dryer as a heat source;
(v-a) rinsing hair with tap water;
(vi) optionally treating hair with a post-treatment;
(iv) reshaping or straightening hair with heat and reshaping aids.

17. Method for semi-permanent hair straightening or semi-permanent hair reshaping according to claim 14, wherein the heat source is selected from hood dryer, hairdryer and hair straightener.
